(19) United States
(12) Patent Application Publication
(10) Pub. No.: US 2024/0277604 A1
(43) Pub. Date: Aug. 22, 2024

(54) SOLID AND POWDERY FORMULATIONS

(71) Applicant: BEIERSDORF AG, Hamburg (DE)

(72) Inventors: Eefje KOOP, Norderstedt (DE); Wiebke SCHLUESCHE, Halstenbek (DE); Cecile RATSCHOW, Hamburg (DE)

(21) Appl. No.: 18/567,427

(22) PCT Filed: May 10, 2022
(86) PCT No.: PCT/EP2022/062570

§ 371 Date: Dec. 6, 2023

(30) Foreign Application Priority Data
Jun. 22, 2021 (JP) 10 2021 206 380.2

(51) Int. Cl. A61K 8/9789; A61K 8/46; A61K 8/73; A61Q 19/10

(52) U.S. Cl. CPC A61K 8/9789; A61K 8/463; A61K 8/731; A61Q 19/10; A61K 2800/87

(57) ABSTRACT

The present invention relates to a cosmetic concentrate for providing a cleansing composition, containing surfactants that are solid under standard conditions, preferably disodium lauryl sulfosuccinates and sodium coco sulfates, and Caesalpinia spinosa gum and/or cellulose gum and preferably also maltodextrin.

Cosmetic products generally not only contribute to a beautiful and attractive appearance, but their effects make a crucial contribution to increased self-esteem and people's wellbeing. Accordingly, a wide variety of cosmetic products are used for daily cleansing and care of human skin.

Conventional cleansing formulations are usually sold in the form of a liquid, aqueous formulation. If the ingredients of such a formulation are analyzed, it can be established that the formulation comprises a significant amount of water. Typical examples of shower gels comprising 20% by weight to 95% by weight water are disclosed in WO1994010975A1, WO1993021900A1 and WO2002005758A2.

Accordingly, significant amounts of water are transported from the supplier to the stores and on to the customer to provide the customer with the cleansing formula of their choice. Considering the fact that most customers' homes are connected to the public water supply, it does not seem sensible from an ecological point of view to send water to a place that in principle has unlimited access to water. By dispensing with water in the formulations and shipping a concentrate in liquid or solid form, transport costs and transport-related emissions of greenhouse gases can thus be reduced.

Novel concepts include, for example, the dispatch of concentrates and mixing with water at the point of sale. Other approaches use solid cleansing compositions in the form of a bar. The latter formulations are applied by the customer like a bar of soap, for example by rubbing the bar over the body. However, the use of a solid bar for applying active cleansing ingredients to the human body is often not desirable for customers who are accustomed to applying cleansing formulations in liquid form.

Consequently, environmentally friendly cosmetic cleansing preparations may also be supplied to the consumer in the form of concentrates. The consumer dissolves the concentrates in water in predetermined proportions, so that a cosmetic cleansing preparation is produced. This form of application saves water in the production of concentrates. This leads to a lower environmental impact by minimizing weight, as the consumer can usually add water at home.

The concentrate may be in liquid or solid form. However, in the case of a solid form, a distinction has to be made between pulverulent preparations, which are free-flowing, and preparations which are in the form of a single, solid piece. The solid piece can be obtained by compression of a pulverulent preparation or by melting and cooling the preparation to give a solidified cohesive structure.

The present application predominantly relates to concentrates in solid form. The aim is that, after mixing with water, the consumer obtains a cleansing preparation that can be foamed by means of a squeeze foamer and, when applied for cleansing, imparts the customary washing effect and creaminess. Creaminess requires that polymers which slightly increase the viscosity compared to water are incorporated into the preparation. A well-known biopolymer often present in cleansing preparations is Xanthan Gum.

The problem is, however, that pulverulent cosmetic cleansing compositions containing Xanthan Gum often exhibit instability after being dissolved in water by the consumer, especially with surfactants that are pulverulent and solid under standard conditions and precipitates occur or there is an uneven thickening of the composition. This disadvantage is to be addressed by the present invention.

Surprisingly, it has now been found that it was possible to eliminate or alleviate the disadvantages of the prior art by means of the present invention.

The present invention relates to a cosmetic cleansing concentrate in solid form comprising, based on the total weight of the concentrate,
a) one or more surfactants that are solid under standard conditions and
b) Caesalpinia spinosa Gum and/or Cellulose Gum;
   characterized in that the concentrate contains less than 7% of ingredients that are liquid under standard conditions.

The invention further relates to a method for providing a cosmetic cleansing composition comprising the steps of
a) mixing
   i. one or more surfactants that are solid under standard conditions,
   ii. Caesalpinia spinosa Gum and/or Cellulose Gum,
   iii. further additives that are solid under standard conditions, and
   iv. optionally not more than 7% by weight of liquid ingredients, based on the mixture of the components i. to iv.;
for preparing a cosmetic cleansing concentrate;
b) dissolving the cosmetic cleansing concentrate in water to obtain the cosmetic cleansing composition.

If percentages by weight (% by weight) should be given below without reference to a particular composition or specific mixture, then these figures always refer to the total weight of the cleansing concentrate. If ratios of components/substances/substance groups should be disclosed below, these ratios refer to ratios by weight of the components/substances/substance groups specified.

If ranges of percentages by weight are given below for the constituents of the cleansing concentrate, the disclosure of the present application also includes all individual values in steps of 0.1% by weight within these weight percentage ranges.

The expressions “according to the invention”, “advantageous according to the invention”, “advantageous in the context of the present invention” etc. in the context of the present disclosure always relate to both the cleansing concentrate according to the invention and to the use according to the invention and the method.

All experiments were carried out under standard conditions unless stated otherwise. The expression “standard conditions” signifies 20° C., 1013 hPa and a relative humidity of 50%.

If the term skin is used, this preferably refers to human skin.

All substances cited below are named by their INCI declaration or by a unique chemical name.

Emulsifiers are understood to mean all substances which are listed in the International Cosmetic Ingredient Dictionary and Handbook, Thirteenth Edition 2010, (ISBN 1-882621-47-6) under the designation “emulsifying agent”. Surfactants are understood to mean all substances which are listed in the International Cosmetic Ingredient Dictionary and Handbook, Thirteenth Edition 2010, (ISBN 1-882621-47-6) under the designation “surfactant”.

Where viscosity values are given in this disclosure, all values refer to a measurement at 25° ° C. in a 150 ml wide-neck bottle (VWR No.: 807-001) using a Rheomat R 123 from proRheo. The Rheomat R 123 from proRheo GmbH is a rotational viscometer, i.e. a measurement body rotates in the substance to be measured. The force is measured that is required to rotate the measurement body in the sample at a predefined speed. The viscosity is calculated from this torque, the speed of the measurement body and the geometric dimensions of the measuring system used. The measurement body used is measurement body No. 1 (article no. 200 0191), suitable for a viscosity range up to 10 000 [mPa·s], rotational speed range 62.5 min⁻¹.

According to the invention, the cosmetic cleansing concentrate comprises at least one surfactant that is solid under standard conditions. Advantageous surfactants are selected from the group Sodium Myristoyl Glutamate, Sodium Lauroyl Glutamate, Sodium Stearoyl Glutamate, Sodium Cocoyl Glutamate, Sodium Coco Sulfate, Disodium Lauryl Sulfosuccinate and Sodium Methyl Cocoyl Taurate. It is particularly advantageous if Disodium Lauryl Sulfosuccinate and/or Sodium Coco Sulfate are present in the cleansing concentrate.

It is advantageous in the context of the present invention if the proportion of surfactants that are solid under standard conditions is from 0.1 to 50% by weight, preferably from 15 to 45% by weight and particularly preferably from 25 to 44% by weight, based on the total weight of the cleansing concentrate.

It is advantageous in the context of the present invention if the proportion of surfactants that are solid under standard conditions selected from the group Sodium Myristoyl Glutamate, Sodium Lauroyl Glutamate, Sodium Stearoyl Glutamate, Sodium Cocoyl Glutamate, Sodium Coco Sulfate, Disodium Lauryl Sulfosuccinate and Sodium Methyl Cocoyl Taurate is from 0.1 to 50% by weight, preferably from 15 to 45% by weight and particularly preferably from 25 to 44% by weight, based on the total weight of the cleansing concentrate.

It is particularly advantageous in the context of the invention if the proportion of Disodium Lauryl Sulfosuccinate and/or Sodium Coco Sulfate is from 0.1 to 50% by weight, preferably from 15 to 45% by weight, preferably from 25 to 44% by weight and particularly preferably from 30 to 43% by weight, based on the total weight of the cleansing concentrate.

Disodium Lauryl Sulfosuccinate is advantageously present at a proportion of 8 to 30% by weight, preferably 10 to 25% by weight and particularly preferably from 12 to 20% by weight, based on the total weight of the cleansing concentrate.

Sodium Coco Sulfate is advantageously present at a proportion of 12 to 34% by weight, preferably 14 to 30% by weight and particularly preferably from 18 to 25% by weight, based on the total weight of the cleansing concentrate.

Furthermore, it is advantageous if Disodium Lauryl Sulfosuccinate and Sodium Coco Sulfate are present and further advantageously the ratio by weight of Disodium Lauryl Sulfosuccinate to Sodium Coco Sulfate is from 1.5:1 to 1:2, preferably 1:1 to 1:1.5 and particularly preferably from 1:1.1 to 1:1.4.

Furthermore, in accordance with the invention, Caesalpinia spinosa Gum and/or Cellulose Gum are present. The total proportion of Caesalpinia spinosa Gum and/or Cellulose Gum is advantageously from 0.1 to 30% by weight, preferably from 0.2 to 25% by weight, more preferably from 0.3 to 20% by weight and particularly preferably 10 to 20% by weight, based on the total weight of the concentrate.

If Caesalpinia spinosa Gum is present, the proportion of Caesalpinia spinosa Gum is advantageously from 0.1 to 30% by weight, preferably from 0.2 to 25% by weight, more preferably from 0.25 to 20% by weight and particularly preferably from 0.3 to 15% by weight, based on the total weight of the concentrate.

If Cellulose Gum is present, the proportion of Cellulose Gum is advantageously from 0.1 to 30% by weight, preferably from 0.2 to 25% by weight, more preferably from 0.25 to 20% by weight and particularly preferably from 0.3 to 15% by weight, based on the total weight of the concentrate.

While both polymers enable a lump-free/precipitation-free preparation when the concentrate is dissolved in water, it was surprisingly found that the exclusive use of Caesalpinia spinosa Gum achieves a particularly uniform thickening.

Accordingly, embodiments comprising Caesalpinia spinosa Gum are preferred in which further polymers are advantageously present at proportions of less than 10% by weight. It is particularly advantageous if only Caesalpinia spinosa Gum is present.

It is further preferable if the concentrate is free from Xanthan Gum, Sphingomonas Ferment Extract, Dehydroxanthan Gum and/or Hydroxypropyl Guar.

Furthermore, it is preferred if the concentrate is free from polymers obtained from a homo- or copolymerization with vinyl pyrrolidone, acrylic and/or methacrylic acid. Such polymers no longer meet today's ecological requirements, since biodegradability thereof has not been conclusively clarified.

In the context of the present invention, “free from” signifies that the total proportion of said substances is less than 0.05% by weight and particularly preferably 0% by weight, the figures referring to the total weight of the concentrate.

Furthermore, it has been shown, surprisingly, that the addition of Maltodextrin significantly increases the creaminess of a cleansing product produced from the concentrate after foaming with a squeeze foamer, without causing precipitates in the cleansing product.

Accordingly, it is advantageous if the concentrate comprises Maltodextrin. If Maltodextrin is present, it is further advantageous if the proportion of Maltodextrin is from 0.1 to 20% by weight, preferably from 2 to 14% by weight and particularly preferably from 3 to 9% by weight, based on the total weight of the concentrate.

Furthermore, it is advantageous if the concentrate comprises Magnesium Carbonate Hydroxide. If Carbonate Hydroxide is present, it is further advantageous if the proportion of Carbonate Hydroxide is from 0.5 to 15% by weight, preferably from 1 to 8% by weight and particularly preferably from 2 to 6% by weight, based on the total weight of the concentrate.

Furthermore, it is advantageous if the concentrate comprises Sodium Benzoate. If Sodium Benzoate is present, it is further advantageous if the proportion of Sodium Benzoate is from 0.1 to 30% by weight, preferably from 5 to 25% by weight and particularly preferably from 10 to 20% by weight, based on the total weight of the concentrate.

Furthermore, it is advantageous if the concentrate comprises Hydroxyacetophenone. If Hydroxyacetophenone is present, it is further advantageous if the proportion of Hydroxyacetophenone is from 0.1 to 12% by weight, preferably from 1 to 10% by weight and particularly preferably from 3 to 7% by weight, based on the total weight of the concentrate.

Furthermore, it is advantageous if the concentrate comprises Citric Acid. If Citric Acid is present, it is further advantageous if the proportion of Citric Acid is from 0.1 to 30% by weight, preferably from 5 to 25% by weight and particularly preferably from 10 to 23% by weight, based on the total weight of the concentrate.

Furthermore, it is advantageous if the concentrate comprises Sodium Citrate. If Sodium Citrate is present, it is further advantageous if the proportion of Sodium Citrate is from 0.1 to 10% by weight, preferably from 1 to 5% by weight and particularly preferably from 2 to 4% by weight, based on the total weight of the concentrate.

Furthermore, it is advantageous if the concentrate comprises DMDM Hydantoin, Chlorphenesin, iodopropyl butylcarbamate and/or Hexamidine Diisethionate.

If DMDM Hydantoin is present, it is advantageous if the proportion of DMDM Hydantoin is from 0.5 to 12% by weight, based on the total weight of the concentrate.

If Chlorphenesin is present, it is advantageous if the proportion of Chlorphenesin is from 0.5 to 13% by weight, based on the total weight of the concentrate.

If iodopropyl butylcarbamate is present, it is advantageous if the proportion of iodopropyl butylcarbamate is from 0.1 to 13% by weight, based on the total weight of the concentrate.

If Hexamidine Diisethionate is present, it is advantageous if the proportion of Hexamidine Diisethionate is from 0.1 to 5% by weight, based on the total weight of the concentrate.

It is advantageous if the proportion of additives that are liquid under standard conditions is less than 7% by weight, preferably less than 6.85% by weight and especially less than 6.5% by weight, based on the total weight of the concentrate.

If water is present as liquid additive in the concentrate, the proportion of water is preferably less than 5% by weight and particularly preferably less than 2% by weight, based on the total weight of the concentrate. This water may be introduced in particular via ingredients that are not completely water-free. Accordingly, it is particularly advantageous if no free water is added to the concentrate.

Free water is water that is added in pure form, i.e. without being added as a constituent of other ingredients.

It is further advantageous if the concentrate comprises one or more perfumes. In the case of liquid perfumes, it is preferable if the proportion of liquid perfumes does not account for more than 7% by weight, based on the total weight of the concentrate.

Furthermore, the concentrate may advantageously comprise further ingredients, such as advantageously colorants or active ingredients. Advantageously, no water-insoluble ingredients are present under standard conditions.

Furthermore, the concentrate is preferably free from soaps. It is preferable if no further surfactants are present.

To provide the cosmetic product, the cleansing concentrate is mixed with water. Advantageously, the ratio by weight of water and concentrate is in the range from 90:1 to 99:1, more preferably from 92:8 to 98:2 and most preferably from 93:7 to 97:3.

The concentrate according to the invention may be present in various embodiments. Thus, it is advantageous according to the invention if the solid concentrate is compressed into a tablet under pressure. Such a format has the advantage that the tablet can easily be added to water. No solid powder is lost due to spillage. Consequently, such a format is advantageous in many respects for use by the consumer at home. Consequently, the concentrate is advantageously in tablet form.

Furthermore, it has been shown that, surprisingly, even as a compressed tablet, the dissolution property of the concentrate according to the invention is particularly advantageous. Thus, the tablet dissolves particularly quickly in water without leaving residues.

The concentrate of the invention is advantageously characterized in that it is in the form of a compressed tablet.

Furthermore, the invention also relates to a cosmetic product comprising a packaging and the concentrate according to the invention, wherein the packaging encloses the concentrate according to the invention. In this case, the concentrate may be in the form of a free powder or compressed tablet.

In a first embodiment, the packaging of the concentrate comprises at least one section consisting of a water-soluble film and at least one section consisting of a material which is not water-soluble. A preferred example of such packaging is a cap for a bottle, wherein the cap is designed such that the section with the water-soluble film is inside the bottle once the cap has been placed onto a bottle. In this manner, the cap may be used on a bottle filled with tap water. Once the cap is placed on the bottle and water comes into contact with the water-soluble film, the concentrate according to the invention is released into the interior of the bottle to form a cleansing composition to be used by the customer.

It is preferred if the closure is designed so that it can be removed from the bottle, either to have the possibility to remove the cleansing composition from the bottle for use, or to allow replacement of the closure so that the bottle can be reused. In this way, transport costs can be reduced, since only the cap is sold to the customer, while the bottle can be reused.

It is further preferred if the cap has a thread so as to be affixed to the bottle in a removable way. Furthermore, it is preferred if the cap which encloses the concentrate is designed such that the cap has a closable opening which is aligned in the cap such that, in the case that the cap is attached to a bottle, the contents of the bottle can be removed. Furthermore, the cap is preferably characterized in that it has a removable seal attached to the cap in such a way that it does not allow water to come into contact with the water-soluble film. This prevents the concentrate from being accidentally released if the packaging gets wet during transport or at home. The seal can be removed by hand or, in preferred embodiments, the seal is attached to the cap in such a way that it automatically breaks when the cap is placed on a suitable bottle.

In another embodiment, the packaging is also a cap, as in the embodiment described above. However, this embodiment differs in that the concentrate is enclosed in the cap by a film that tears when the cap is placed on the bottle, so that the concentrate is automatically dispensed into the interior of the bottle.

In a further embodiment, the concentrate of the invention is enclosed by the packaging, the packaging consisting of a film which is water-soluble. Accordingly, the cosmetic product in this embodiment can be added to a bottle filled with water, in which the film dissolves and releases the concentrate.

According to the invention, the term “water-soluble film” is understood to mean a film that breaks as soon as the film is surrounded by water.

It is preferred if the water-soluble film described in the above embodiments comprises or consists of polyvinyl alcohol, gelatin and/or alginates, with polyvinyl alcohol being preferred.

In a further embodiment, the concentrate is present as powder in a packaging, with the packaging being biodegradable. Accordingly, the packaging preferably does not comprise plastic.

If the concentrate is mixed with water in a bottle, the cleansing composition is advantageously applied according to the invention in such a way that the cleansing composition is withdrawn by means of a hand-operated pump piston. In particular, suitable pump pistons advantageously allow foaming of the cleansing composition.

### COMPARATIVE EXPERIMENTS AND EXAMPLES

The examples below are intended to illustrate the present invention without limiting it. Unless stated otherwise, all quantitative data, fractions, and percentages are based on the weight and the total amount or on the total weight of the preparations.

The following tables show various concentrates and observations.

Viscosity was measured as stated in the description above. Ex. 1 to Ex. 4 are comparative examples. Ex. 5 to Ex. 8 are according to the invention.

| Ingredients | Ex. 1 | Ex. 2 | Comp. 1 | Comp. 2 | Comp. 3 |
|---|---|---|---|---|---|
| Citric Acid | 15.63 | 15.63 | 15.63 | 15.63 | 15.63 |
| Sodium Benzoate | 15.63 | 15.63 | 15.63 | 15.63 | 15.63 |
| 99% Sodium Citrate + | 2.34 | 2.34 | 2.34 | 2.34 | 2.34 |
| 1% Aqua | | | | | |
| Magnesium Carbonate | 2.34 | 2.34 | 2.34 | 2.34 | 2.34 |
| Hydroxide | | | | | |
| 95% Disodium Lauryl | 15.63 | 15.63 | 15.63 | 15.63 | 15.63 |
| Sulfosuccinate + 5% | | | | | |
| Aqua | | | | | |
| Maltodextrin | 7.81 | 7.81 | 7.81 | 7.81 | 7.81 |
| Hydroxyacetophenone | 5.47 | 5.47 | 5.47 | 5.47 | 5.47 |
| 97% Sodium Coco | 19.52 | 19.52 | 19.52 | 19.52 | 19.52 |
| Sulfate + 3% Aqua | | | | | |
| Caesalpinia Spinosa | 15.63 | | | | |
| Gum | | | | | |
| Cellulose Gum | | 15.63 | | | |
| Dehydroxanthan gum | | | 15.63 | | |
| Hydroxypropyl Guar | | | | 15.63 | |
| Xanthan gum | | | | | 15.63 |

The concentrates listed above were mixed with water at a ratio by weight of 14.625:1. For mixing, these were shaken for 20 seconds. The dissolution and thickening properties were assessed 24 hours later:

| Ingredients | Ex. 1 | Ex. 2 | Comp. 1 | Comp. 2 | Comp. 3 |
|---|---|---|---|---|---|
| Lumps/ | No | No | Dehydroxanthan | Undissolved | Undissolved |
| Precipitates | small | small | Gum settles, | powder | powder |
| | lumps | lumps | phase | lumps | lumps |
| | | | separation | | |
| Thickening | Uniformly | Thickened | Water thin | Water thin | Water thin |
| | thickened | only at the | | | |
| | | top, | | | |
| | | remainder | | | |
| | | water thin | | | |

Further examples according to the invention are shown below:

| Ingredient | Ex. 3 | Ex. 4 | Ex. 5 | Ex. 6 | Ex. 7 | Ex. 8 |
|---|---|---|---|---|---|---|
| Citric Acid | 23.87 | 25.64 | 16.65 | 20.80 | 19.67 | 21.65 |
| Sodium Benzoate | 20.06 | 15.54 | 18.34 | 17.33 | 17.95 | 15.64 |
| 99% Sodium Citrate + 1% | 3.80 | 1.30 | 4.86 | 2.60 | 3.97 | 3.64 |
| Aqua | | | | | | |
| Magnesium Carbonate | 3.55 | 8.30 | 6.63 | 5.20 | 4.86 | 4.80 |
| Hydroxide | | | | | | |
| 95% Disodium Lauryl | 16.05 | 13.64 | 17.88 | 17.33 | 10.67 | 20.67 |
| Sulfosuccinate + 5% Aqua | | | | | | |
| Maltodextrin | 3.85 | 1.66 | 5.34 | 3.47 | 6.84 | 4.09 |
| Hydroxyacetophenone | 5.93 | 2.82 | 4.37 | 6.07 | 6.90 | 7.49 |
| 97% Sodium Coco Sulfate + | 15.88 | 22.34 | 20.46 | 21.65 | 23.59 | 17.37 |
| 3% Aqua | | | | | | |
| Caesalpinia Spinosa Gum | 1.00 | 0.76 | 0.15 | 0.35 | 0.65 | 0.70 |
| Fragrance | 5.90 | 6.20 | 4.55 | 5.20 | 4.90 | 3.95 |
| Aqua | to | to | to | to | to | to |
| | 100 | 100 | 100 | 100 | 100 | 100 |

1.-17. (canceled)

18. A cosmetic cleansing concentrate, wherein the concentrate is present in solid form and comprises
(a) one or more surfactants that are solid under standard conditions and
(b) Caesalpinia spinosa Gum and/or Cellulose Gum;
the concentrate comprising less than 7% by weight, based on a total weight of the concentrate, of ingredients that are liquid under standard conditions.

19. The concentrate of claim 18, wherein the one or more solid surfactants comprise one or more of Sodium Myristoyl Glutamate, Sodium Lauroyl Glutamate, Sodium Stearoyl Glutamate, Sodium Cocoyl Glutamate, Sodium Coco Sulfate, Disodium Lauryl Sulfosuccinate and Sodium Methyl Cocoyl Taurate.

20. The concentrate of claim 18, wherein the concentrate comprises Disodium Lauryl Sulfosuccinate and/or Sodium Coco Sulfate.

21. The concentrate of claim 18, wherein the concentrate comprises from 0.1% to 50% by weight of (a), based on the total weight of the concentrate.

22. The concentrate of claim 21 wherein the concentrate comprises from 15% to 45% by weight of (a).

23. The concentrate of claim 18, wherein the concentrate comprises from 8% to 30% by weight of Disodium Lauryl Sulfosuccinate, based on the total weight of the concentrate.

24. The concentrate of claim 18, wherein the concentrate comprises from 12% to 34% by weight of Sodium Coco Sulfate, based on the total weight of the concentrate.

25. The concentrate of claim 18, wherein the concentrate comprises Disodium Lauryl Sulfosuccinate and Sodium Coco Sulfate at a weight ratio of from 1.5:1 to 1:2.

26. The concentrate of claim 18, wherein the concentrate comprises a total of from 0.1% to 30% by weight of Caesalpinia spinosa Gum and/or Cellulose Gum, based on the total weight of the concentrate.

27. The concentrate of claim 18, wherein the concentrate comprises a total of from 0.2% to 25% by weight of Caesalpinia spinosa Gum and/or Cellulose Gum, based on the total weight of the concentrate.

28. The concentrate of claim 18, wherein the concentrate is free from Xanthan Gum, Sphingomonas Ferment Extract, Dehydroxanthan Gum and Hydroxypropyl Guar.

29. The concentrate of claim 18, wherein the concentrate further comprises maltodextrin.

30. The concentrate of claim 29, wherein the concentrate comprises from 0.1% to 20% by weight of maltodextrin, based on the total weight of the concentrate.

31. The concentrate of claim 18, wherein the concentrate further comprises sodium benzoate.

32. The concentrate of claim 18, wherein the concentrate comprises less than 6.85% by weight of ingredients that are liquid under standard conditions.

33. The concentrate of claim 18, wherein the concentrate comprises less than 5% by weight of water, based on the total weight of the concentrate.

34. The concentrate of claim 18, wherein the concentrate is present in the form of a tablet or a powder.

35. A cosmetic product, wherein the product comprises the concentrate of claim 18 and a packaging enclosing the concentrate.

36. The product of claim 35, wherein the packaging has at least one section that consists of a water-soluble film.

37. A method for providing a cosmetic cleansing composition, wherein the method comprises
(a) mixing
i. one or more surfactants that are solid under standard conditions,
ii. Caesalpinia spinosa Gum and/or Cellulose Gum,
iii. further additives that are solid under standard conditions, and
iv. optionally, not more than 7% by weight of liquid ingredients, based on a total weight of i. to iv.;
to prepare a cosmetic cleansing concentrate;
(b) dissolving the cosmetic cleansing concentrate in water to obtain the cosmetic cleansing composition.
